(19) United States
(12) Patent Application Publication
(10) Pub. No.: US 2015/0151799 A1
(43) Pub. Date: Jun. 4, 2015

(54) MOBILE STERILIZATION PROCESSING FACILITY

(71) Applicant: Mobile Medical International Corporation, St. Johnsbury, VT (US)

(72) Inventors: Philip T. Cantin, Guildhall, VT (US); James T. Isherwood, JR., Littleton, NH (US)

(73) Assignee: MOBILE MEDICAL INTERNATIONAL CORPORATION, St. Johnsbury, VT (US)

(21) Appl. No.: 14/321,741

(22) Filed: Jul. 1, 2014

Related U.S. Application Data
(60) Provisional application No. 61/841,620, filed on Jul. 1, 2013.

(51) Int. Cl. B62D 53/06; A61L 2/00; B62D 39/00

(52) U.S. Cl. CPC B62D 53/067; B62D 39/00; A61L 2/00

(57) ABSTRACT

The present technology relates generally to an expandable shelter system, and more particularly to a mobile expandable shelter system with a sterilization processing facility. In embodiments, the shelter system comprises, for example, a mobile shelter comprising a decontamination area with a decontamination area air pressure, a sterilization area physically segregated from the decontamination area with a sterilization area air pressure; at least one wall configured to isolate the sterilization area from the decontamination area, and an HVAC system configured to keep decontamination area air pressure lower than sterilization area air pressure.

### CROSS-REFERENCE TO RELATED APPLICATIONS

This application claims the benefit of U.S. Provisional Patent Application No. 61/841,520, filed Jul. 1, 2013. The content of these applications is hereby incorporated by reference herein.

### BACKGROUND

1. Field of the Technology

The present technology relates generally to an expandable shelter system, and more particularly to a mobile expandable shelter system with a sterilization processing facility.

2. Related Art

Portable shelters are often used to provide temporary facilities for various purposes, such as military, civilian, and medical applications. Such portable shelters may be used to supplement permanent structures when additional space is desired, or to provide new facilities for temporary use, such as the provision of emergency response services after a disaster. Motorized vehicles, such as vans, buses, and recreational vehicles (RVs), etc., may be used as portable shelters under certain circumstances. While these types of motorized vehicles are able to transport themselves to a desired location, they may provide limited interior space for intended use, while also being relatively expensive.

The floor space of conventional portable shelters is limited by the fixed external dimensions of the shelter. Expansion modules akin to “slide out” sections of RVs have been used to increase the floor space enclosed by a shelter. Such modules, also known as “expandable components,” may be hydraulically or mechanically driven to extend and retract from the shelter on support beams.

### SUMMARY

In one aspect, there is provided an expandable mobile sterilization shelter, comprising a main, non-expandable shelter body and one or more expandable shelter bodies movable relative to the main shelter body between collapsed and expanded states in which the shelter has relatively less and more available enclosed space, respectively; and a sterilization facility including a decontamination area, processing and packaging area and sterilization area, and sterilization devices for use in each of the sterilization facility areas, wherein the facility and a first set of sterilization devices form a nondivisible load, wherein the mobility and position of the first set of sterilization devices in the facility are such that the weight of the facility is distributed across the main and one or more expandable shelter bodies when the facility is in a mobile layout when the shelter is in its collapsed state, and when the facility is in its operational layout when the shelter is in its expanded state.

In another aspect, there is provided an expandable mobile sterilization shelter, comprising: a main non-expandable shelter body; first and second expandable shelter bodies; at least two shelter components; a non-divisible load including sterilization components, subset of shelter components, desired/valued for commercial use of sterilization; and other components that are manually removable.

In another aspect, there is provided an expandable mobile sterilization shelter, comprising: a main non-expandable shelter body (always there); first and second expandable shelter bodies; wherein when in operational layout provides decontamination, packaging and sterilization areas and satisfies standard sterilization, mobile medical unit standards; wherein when in mobile layout, satisfies transportation standards.

In another aspect, there is provided a mobile shelter, comprising: a decontamination area with a decontamination area air pressure; a sterilization area physically segregated from the decontamination area with a sterilization area air pressure; at least one wall configured to isolate the sterilization area from the decontamination area; and an HVAC system configured to keep decontamination area air pressure lower than sterilization area air pressure.

In another aspect, there is provided a mobile sterilization processing shelter, comprising: a sterilization area with a sterilizer module and a housing for sterilizer module configured to contain excess heat released by sterilizer module; a steam generator area with a steam generator module and a housing for steam generator module configured to contain excess heat released by steam generator module; and a ventilation system configured to remove excess heat from sterilizer housing and steam generator housing.

In another aspect, there is provided a semi-trailer truck, comprising: a tractor; and a shelter/trailer with a main non-expandable shelter body, first and second expandable shelter bodies, at least two shelter components, and a non-divisible load including sterilization components, subset of shelter components, desired/valued for commercial use of sterilization.

### BRIEF DESCRIPTION OF THE DRAWINGS

Embodiments of the present technology are described below with reference to the attached drawings, in which:

FIG. 1 is a side view of semi-trailer truck having a semi-trailer shelter, according to embodiments of the present technology;

FIG. 2A is a perspective view of an expandable shelter with a main shelter body and two shelter bodies, according to embodiments of the present technology;

FIG. 2B is a rear view of the expandable shelter illustrated in FIG. 2A;

FIG. 2C is a rear schematic view of the expandable shelter illustrated in FIG. 2A;

FIG. 3A is a top view of the sterilization processing facility, according to embodiments of the present invention;

FIG. 3B is a top view of the sterilization processing facility illustrated in FIG. 3A in its mobile, or transportable, layout, according to embodiments of the present invention;

FIG. 3C is a schematic diagram of the sterilization processing facility illustrated in FIGS. 3A and 3B;

FIG. 4 illustrates a high-level view of a sterilization processing facility including the internal walls of sterilization processing facility, according to embodiments of the present invention; and

FIG. 5 illustrates a sterilization processing facility in its mobile, or transportable, layout, according to embodiments of the present invention.

The drawings are intended to illustrate aspects of the technology, and as such, are not necessarily to scale and may omit aspects well known to those of skill in the art and aspects not relevant to the disclosed features.

### DETAILED DESCRIPTION

Aspects and embodiments of the present technology are directed to an expandable shelter system with a mobile sterilization processing facility. More specifically, the present technology includes a mobile sterilization processing facility with a preparation and packaging area and a sterilization area (the “clean” areas) and a decontamination area (the “dirty” area), wherein the decontamination area is physically segregated and isolated from the clean areas. Furthermore, the mobile sterilization processing facility complies with federal and state standards when in its expanded, operational layout and also when in its collapsed, mobile layout. Aspects of the present technology are described herein with reference to a mobile shelter system with a mobile sterilization processing facility, within a semi-trailer truck. It should be appreciated, however, that embodiments of the present technology may be implemented in other mobile technologies.

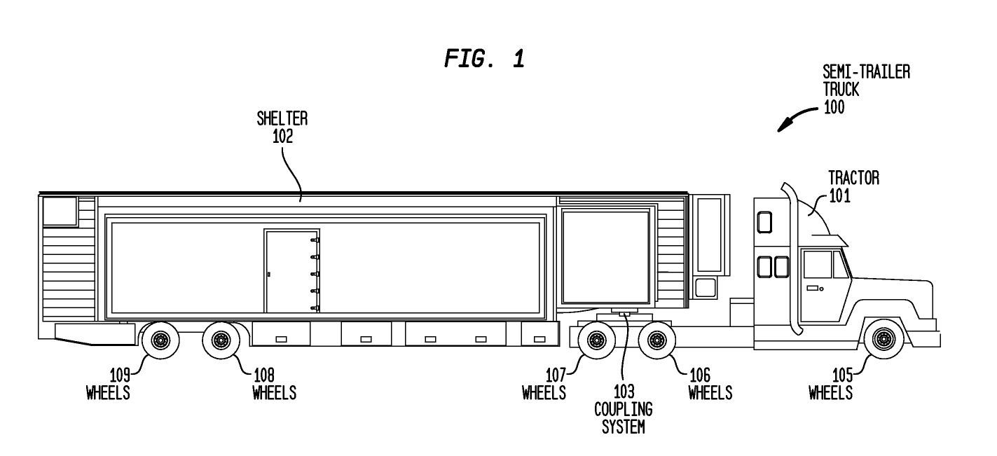

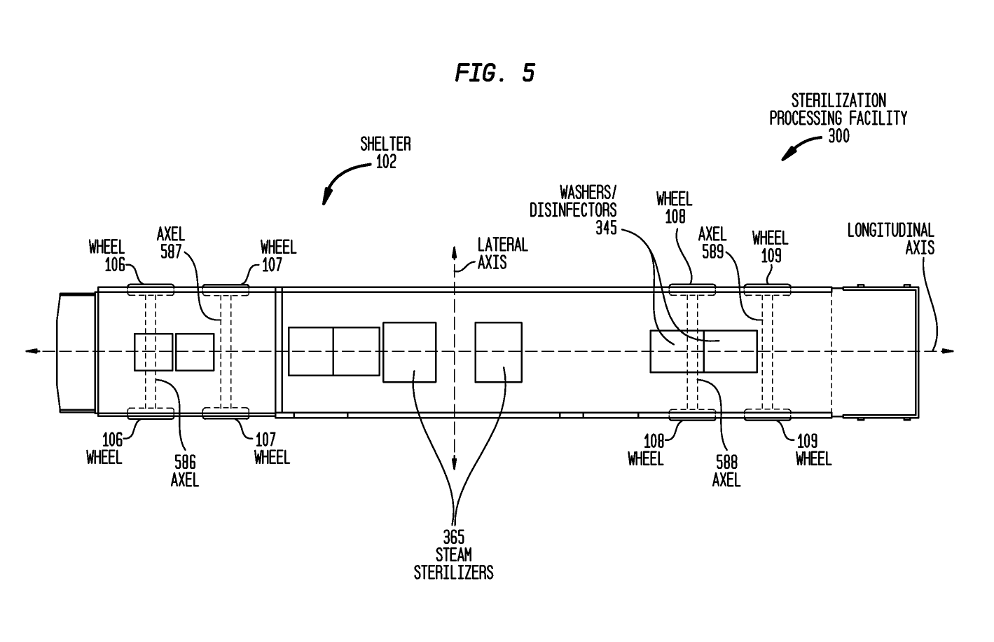

FIG. 1 shows a side view of semi-trailer truck 100. Semi-trailer truck 100 includes a shelter 102, in accordance with embodiments of the present invention. Shelter 102 is shown in FIG. 1 as a semi-trailer, or just “trailer,” which is connected to a tractor, or “towing engine,” 101. Tractor 101 is connected to shelter 100 using a coupling system 103. Coupling system 103 may include a fifth wheel coupling, a kingpin and/or other mechanisms for connecting tractor 101 to shelter 102 such that semi-trailer truck 100 is safe for travel. Tractor 101 includes sets of wheels 105, 106 and 107. Wheels 105 are located towards the front of tractor 101, and 106 and 107 are located towards the rear of tractor 101 and just below the front end of shelter 102. Shelter 102 includes sets of wheels 108 and 109, which are located towards the rear of shelter 102, as shown in FIG. 1. Wheels 105, 106 and 107 are each connected to a set of wheels on the opposite side of tractor 101 and wheels 108 and 109 are each connected to a set of wheels on the opposite side of shelter 102. Each pair of opposing wheels are connected to each other by an axle (not shown). The relationship between the wheels, axles and the rest of the semi-trailer truck 100 is described below with reference to FIG. 5.

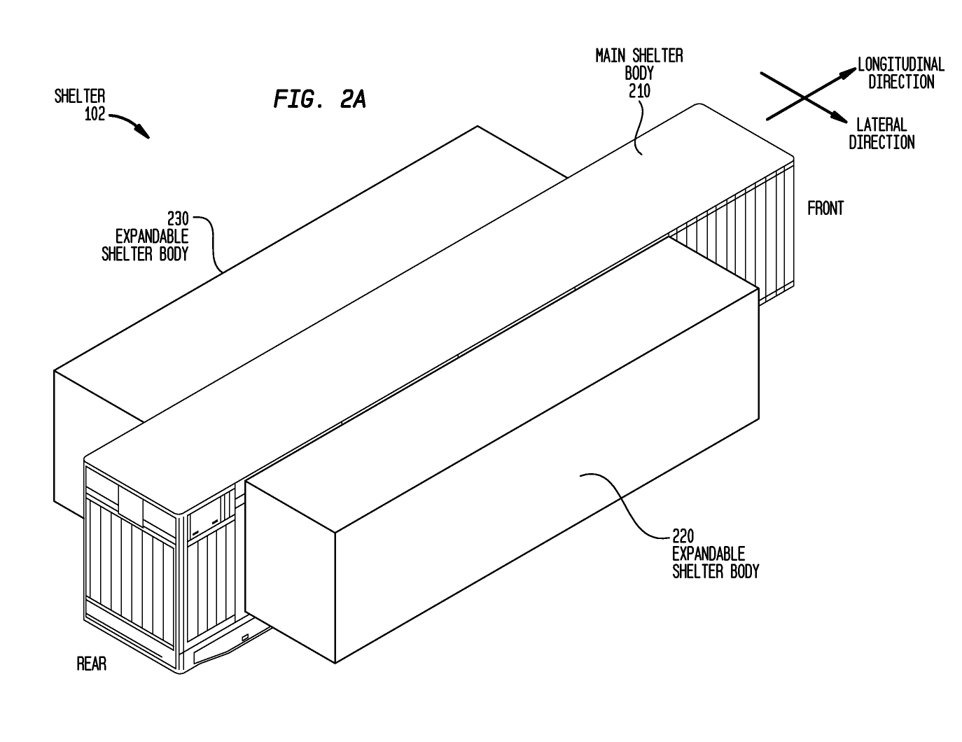

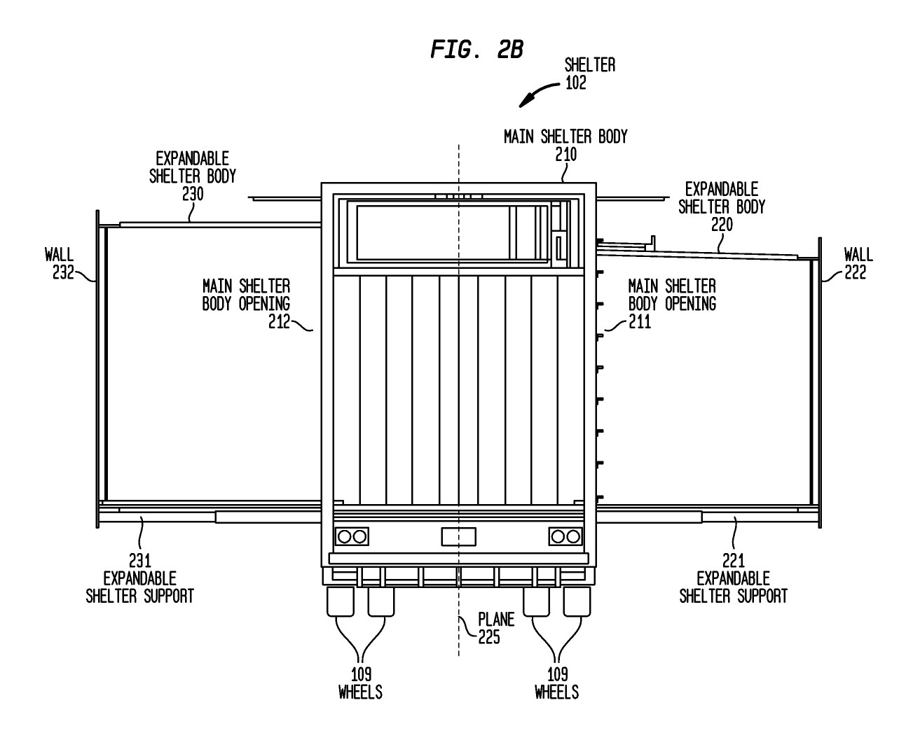

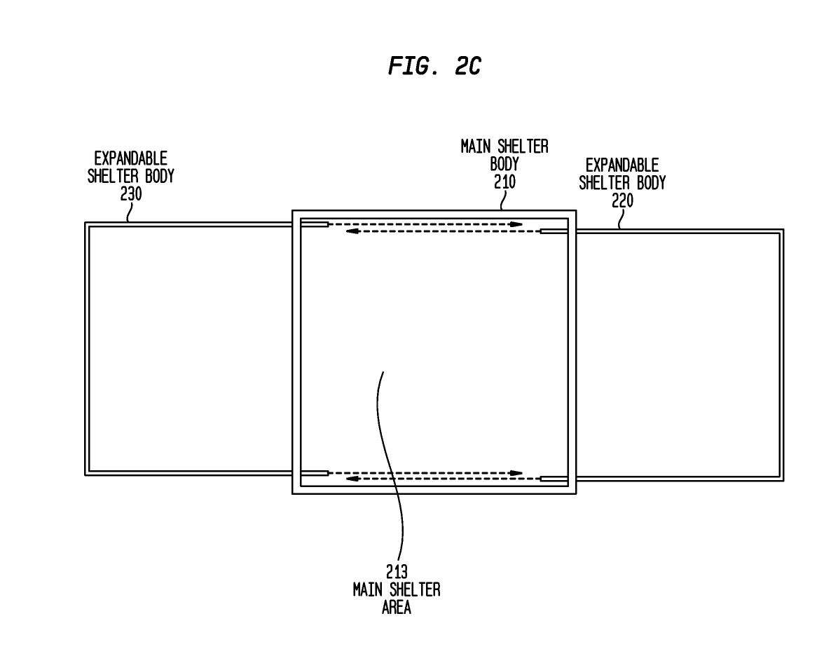

Shelter 100 is mobile as it is capable of being moved by tractor 101 when connected to tractor 101 via coupling system 103. However, when semi-trailer truck 100 is not moving, shelter 102 may be held immobile and expanded using expandable shelter bodies, as described below with respect to FIG. 2A.

FIG. 2A shows a perspective view of a shelter 102. Shelter 102 includes a main non-expandable shelter body 210 and expandable shelter bodies 220 and 230 shown in their extended configuration (which are not shown in FIG. 1 for purposes of clarity). Expandable shelter body 220 is shown in FIG. 2A as extended from one longitudinal side (one side parallel to plane 225) of main shelter body 210 and expandable shelter body 230 is shown in FIG. 2A as extended from the other longitudinal side (also parallel to plane 225 of FIG. 2B) of main shelter body 210.

Expandable shelter bodies 220 and 230, when extended into their expanded state as shown in FIG. 2A, provide for significantly more space within shelter 102 than when they are in a collapsed state (shelter 102 is shown in its collapsed state in FIG. 1, for example). As noted, the floor space of main shelter body 210 is limited by the fixed external dimensions of the main shelter body 210. This provides, in at least some embodiments, an “enclosed space multiplier” effect in that the available enclosed space for the shelter can be quickly expanded from that which might be provided by shelter 102 without these expandable shelter bodies. In one embodiment, as shown in FIG. 2A, expandable shelter bodies 220 and 230 increase the floor space and overall internal capacity of shelter 102 by slightly less than a factor of three. This expansion is possible because, as shown in FIG. 2A, expandable shelter bodies 220 and 230 are almost as long as main shelter body 210 in the longitudinal direction and may be just as wide in the lateral direction.

Expandable shelter bodies 220 and 230 each generally form volumes in the form of rectangular boxes, although in other embodiments, other shaped volumes may be utilized (e.g., the roofs may be sloped away from the main shelter body 110′, the volumes may be square boxes, etc.). These expandable shelter bodies have outer boundaries (e.g., walls, doors, etc.) which, along with main shelter body 210, establish the boundaries of shelter 102.

FIG. 2B shows a rear view of shelter 102. FIG. 2C further depicts expandable shelter bodies 220 and 230. As noted, expandable shelter bodies 220 and 230 extend from and retract into main shelter body 210. More specifically, expandable shelter body 220 and 230 extend and retract in opposite direction normal to the longitudinal vertical plane 225 of the shelter 102. Expandable shelter body 220 extends from and retracts into main shelter body opening 211 and expandable shelter body 230 extends from and retracts into main shelter body opening 212. The retraction of shelter 102 is shown by arrows in FIG. 2C. When shelter 102 is in a fully collapsed state; that is, when and expandable shelter bodies 220 and 230 are fully retracted, walls 222 and 232 of expandable shelter bodies 220 and 230, respectively, will form the side walls of main shelter body 210. When shelter 102 is in this collapsed state, the rest of expandable shelter bodies 220 and 230 will be hidden within main shelter body 210.

According to embodiments of the present invention, shelter 102 includes a plurality of expandable shelter supports, for example, 221 and 231. Each shelter support is shown in an extended configuration. Expandable shelter supports 221 and 231 are powered shelter supports (one or both of which may be unpowered in some embodiments). Each expandable shelter support 221 and 231 includes a drive assembly such as a hydraulic cylinder subassembly configured to extend and retract the telescopic support. Shelter bodies 220 and 230 are not powered in some embodiments (and one or both may be powered in other embodiments, and in other embodiments, none may be powered). In such embodiments, shelter bodies 220 and 230 are extended and retracted as a result of being mechanically linked to expandable shelter supports 221 and 231 (e.g., by being attached to a common structure, such as expandable shelter bodies 220 and 230, and, accordingly, the expandable shelter supports move in unison). Because the ends of expandable shelter supports 221 and 231 travel with mating components of their respective expandable shelter body, the expandable shelter bodies are supported against the direction of gravity generally at their outboard portions by the expandable shelter supports 221 and 231.

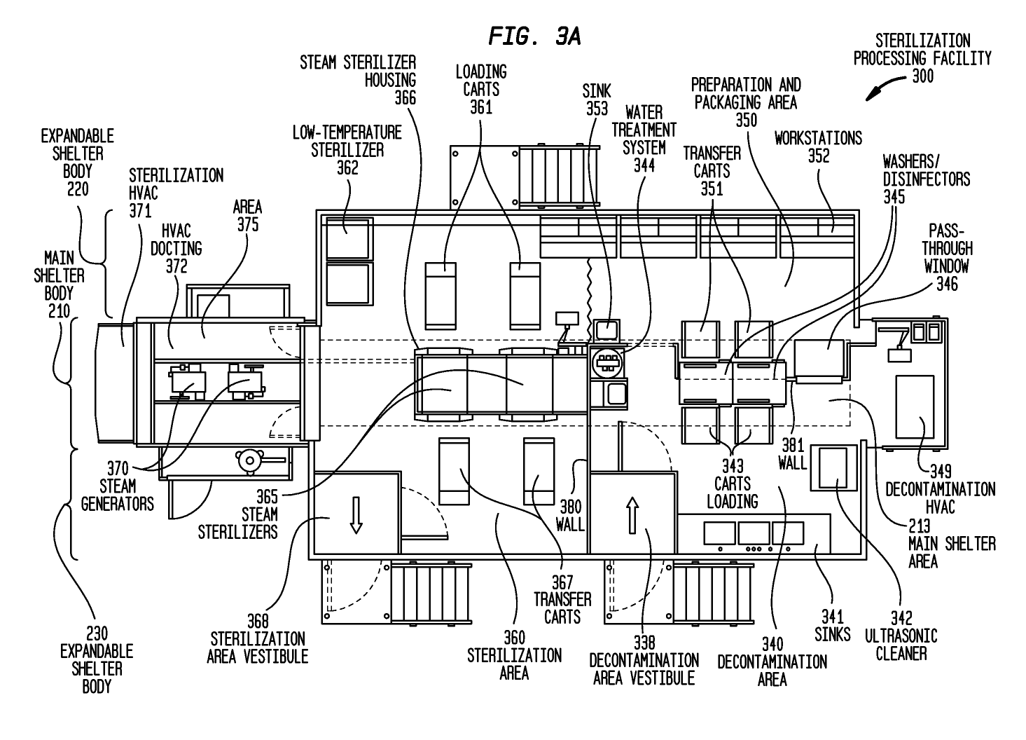

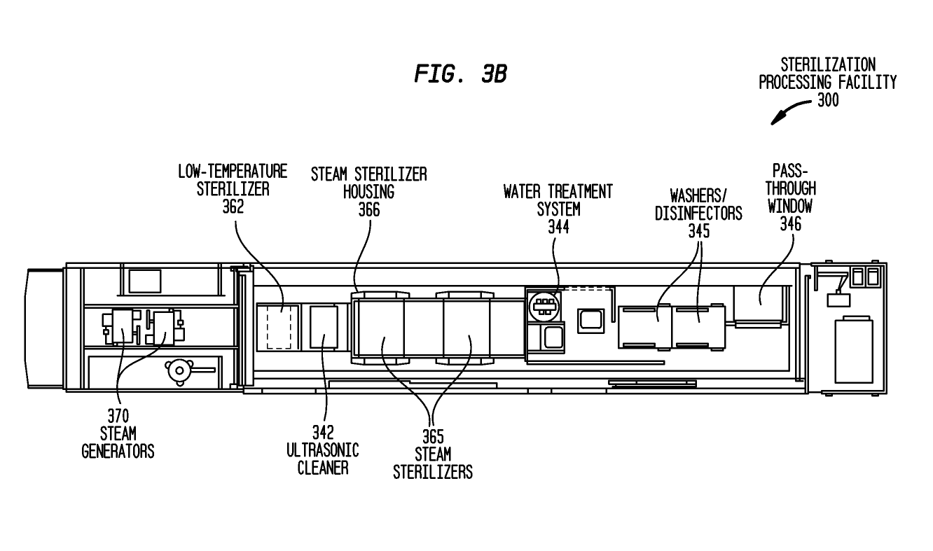

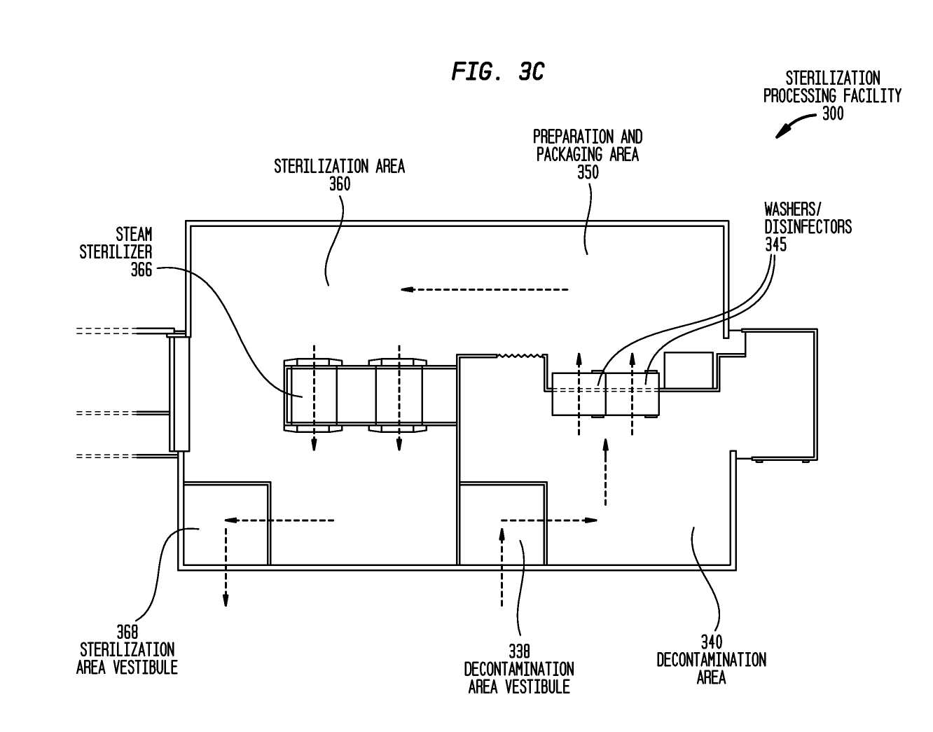

FIG. 3A shows shelter 102 in its expanded state when implemented as a sterilization processing facility 300, according to embodiments of the present technology. As used herein, the phrase “sterilization processing facility” is a facility used to sterilize medical instruments and other items in accordance with the appropriate standards now or later developed, such as, for example, Facilities Guideline Institute (FGI) guidelines for Design and Construction of Health Care Facilities and the ANSI/AAMI ST79 Guide for Steam Sterilization. Sterilization processing facility 300 includes sterilization devices to perform at least the processes of decontamination, preparation/packaging and sterilization. Each stage of the sterilization processing pipeline is implemented with the appropriate safeguards required by the standards—air handling, contamination control, etc. The area of the expanded shelter that is dedicated to the implementation of each stage is based on a variety of factors including the relationship of that stage to other stages in the pipeline, the air handling requirements for each stage, the weight of the equipment used to perform the operations within the stage, the heat and other debris generated by the equipment for each stage, among other factors.

FIG. 3A shows exemplary mobile sterilization processing facility 300 in its operational layout. Sterilization processing facility 300 is in its operational layout when shelter 102, in which sterilization processing facility 300 is housed, is in its expanded state. FIG. 3B, on the other hand, shows sterilization processing facility 300 in its mobile, or transportable (“packed-up”), layout. Sterilization processing facility 300 is in its mobile layout when shelter 102 is in its collapsed state.

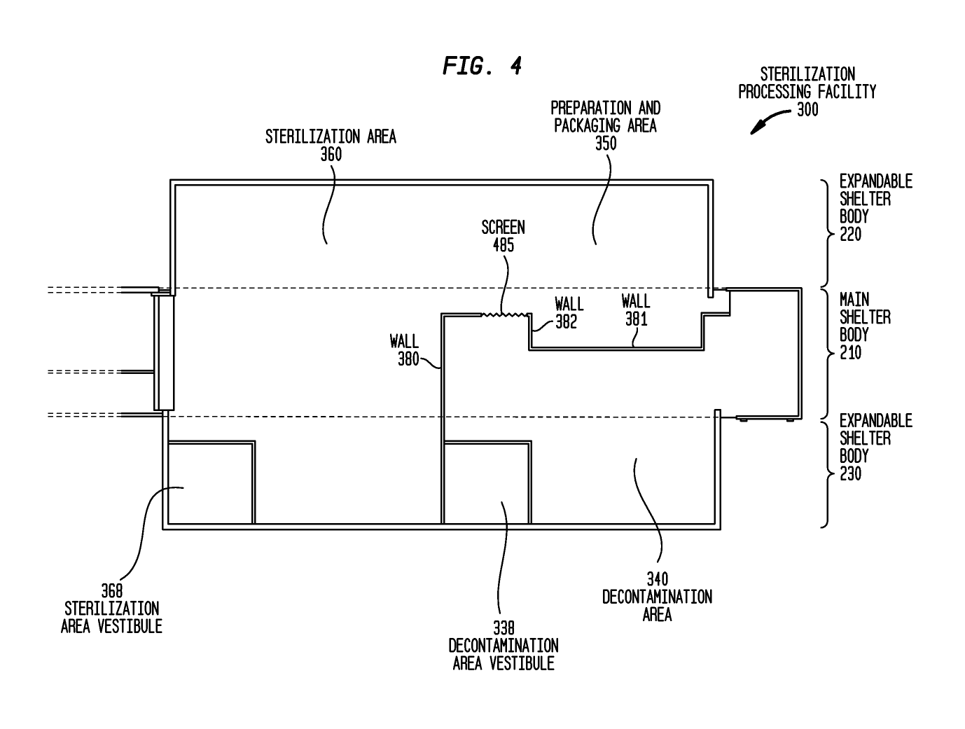

The sterilization processing facility 300 includes at least three main areas, or stages of the sterilization processing pipeline performed by facility 300, that include devices for decontamination, preparation/packaging and sterilization, respectively. For example, sterilization processing facility 300 includes decontamination area 340, preparation and packaging area 350 and sterilization area 360. Preparation and packaging area 350 and sterilization area 360 may be referred to herein as “clean” or the “clean area”; decontamination area 340 may be referred to herein as the “dirty” area. In the embodiment shown in FIG. 3A, sterilization area 360, preparation and packaging area 350 and decontamination area 340 each take up a portion of expanded shelter 102. However, the size and shape of each area are proportional to each other area based on the equipment used in each processing stage. In other words, the structure and scaling of each area/stage is fully customizable and adjustable depending on the use or application of the facility 300. Furthermore, the internal walls within shelter 102, such as for example wall 380, may be removable and adjustable (i.e. foldable), allowing the size of decontamination area 340, preparation and packaging area 350 and sterilization area 360 to be adjusted when being used in different capacities or when more space is required for one or more of these areas. These walls, including wall 380, and their utility are described below with reference to FIG. 4.

Decontamination area 340 includes various devices, or types of equipment, used for decontamination of medical instruments. For example, decontamination area 340 includes water treatment system 344, washers/disinfectors 345, ultrasonic cleaner 342, decontamination area vestibule 338, and sinks 341. Water treatment system 344 and washers/disinfectors 345 are located within the main shelter body 210 of shelter 102, and therefore may be secured in place (e.g. to the wall, floor, etc.). On the other hand, ultrasonic cleaner 342 and sinks 341 are located within expandable shelter body 230 portion of shelter 102. Therefore, when shelter 102 is in its collapsed state and facility 300 is in its mobile layout, ultrasonic cleaner 342 and sinks 341 and any other devices within expandable shelter body 230 are moved into main shelter body 210. Therefore, ultrasonic cleaner 342 and sinks 341 must be movable and not secured in place within the expandable shelter body.

As shown in FIG. 3A, a portion of washers 345 extend through wall 381, which separates decontamination area 340 and preparation and packaging area 350, such that washers 345 are accessible from both decontamination area 340 and preparation and packaging area 350 without requiring the user to walk from one stage to the other. Washers 345 may be, for example, high pressure washers. Washers 345 are double-door devices and are secured within shelter 102 such that one door is located inside decontamination area 340 and the other door is located inside preparation and packaging area 350. Therefore, after an article (which is brought into facility 300 for decontamination and sterilization) is brought to washers 345 via loading carts 343 and loaded into washers 345, the article may be washed and subsequently passed to preparation and packaging area 350 and on to transfer carts 351. If ultrasonic cleaner 342 or decontamination area 340 are used to wash/disinfect the article, then the article may be sent to preparation and packaging area 350 via pass-through window 346.

In addition to washers 345, transfer carts 351 and pass-through window 346, preparation and packaging area 350 also includes workstations 352 and sink 353, and may include various other devices used for preparation and packaging of medical devices before they are transferred to sterilization area 360.

As further shown in FIG. 3A, sterilization area 360 includes various devices used for sterilization of medical instruments after they are washed and prepared for sterilization. For example, sterilization area 360 includes steam sterilizers 365, low-temperature sterilizers 362, loading carts 361, transfer carts 367, and sterilization area vestibule 368, among many other possible devices/equipment. Similar to decontamination area 340, sterilization area 360 includes devices that are located within main shelter body 210 and other devices that are located within expandable shelter bodies 220 and 230. For example, as shown in FIG. 3A, steam sterilizers 365 are located within the main shelter body 210, and therefore may be secured in place (e.g. bolted to the floor). On the other hand, low-temperature sterilizers 362, loading carts 361 and transfer carts 367 are located within the expandable shelter bodies 220 and 230 portion of shelter 102. Therefore, when shelter 102 is in its collapsed state and facility 300 is in its mobile layout, low-temperature sterilizers 362, loading carts 361 and transfer carts 367 and any other devices within expandable shelter bodies 220 and 230 must be moved into main shelter body 210. Therefore, low-temperature sterilizers 362, loading carts 361 and transfer carts 367 must be movable and not permanently secured in place within the expandable shelter body.

It should be understood that although certain types of sterilization equipment is shown in FIG. 3A and described herein, various other types of equipment may be included in sterilization area 360. For example, other types of sterilizers or washers may be used rather than the ones specifically mentioned herein and would achieve a similar result.

Sterilization processing facility 300 also includes decontamination HVAC 349, which provides heating, ventilation and air conditioning; i.e., temperature/climate control, to decontamination area 340. HVAC 349 is on the opposite end of shelter 102 as sterilization HVAC 371, which provides temperature/climate control to sterilization area 360 and/or other clean areas of sterilization processing facility 300. HVAC 349 and HVAC 371, which service dirty and clean portions of facility 300, respectively, are separated to prevent contamination of sterilization area 360 with contents or air from decontamination area 340. Such prevention of contamination between decontamination and sterilization areas will be discussed further with respect to FIG. 4.

Sterilization processing facility 300 also includes another area, HVAC area 375, which is located directly above the rear portion of tractor 101 and coupling system 103. HVAC area 375 is segregated from decontamination area 340 and sterilization area 360. HVAC area 375 includes steam generators 370 and may include other devices, such as HVAC ducting 372. Steam generators provide steam to steam sterilizer 365.

As shown in FIG. 3B, when shelter 102 is converted from its expanded state to its collapsed state, and sterilization processing facility 300 is therefore converted from its operational layout to its mobile layout, certain movable devices within facility 300 are moved from expandable shelter bodies 220 and 230 into main shelter body 210. For example, as shown in FIGS. 3A and 3B, ultrasonic cleaner 342 is moved from expandable shelter body 230 to main shelter body 210 and low-temperature sterilizer 362 is moved from expandable shelter body 220 to main shelter body 210. Furthermore, since the space within shelter 102 is reduced by almost two-thirds when converting from expanded state to collapsed state, some devices are removed from facility 300. For example, devices that may be removed from facility 300 when converted into mobile layout are loading carts 343 and 361, transfer carts 351 and 367, sinks 341, workstations 352, ultrasonic cleaner 342, low-temperature sterilizer 362, walls 380 and 381, among others. Removed devices may be transported separately in, for example, a chase vehicle or another method of transportation, or may not travel with shelter 102 at all. Furthermore, when in a collapsed (mobile) state, sterilization processing facility 300 within shelter 102 must meet Federal Motor Vehicle Safety Standards (FMVSS) to be transported as a semi-truck trailer.

FIG. 3C shows a high-level view of sterilization processing facility 300 including a processing pipeline of an exemplary procedure to decontaminate, prepare and package, and sterilize an article such as a medical device. Dirty or soiled articles, such as medical devices, are first brought into decontamination area 340 via decontamination area vestibule 338 (which may be used for storage of soiled articles before they are brought to decontamination area 340 for washing). As noted, the article is decontaminated in decontamination area 340 using a cleaning device, such as washers 345. However, the article may be decontaminated using other methods such as hand-washed using a sink (such as sinks 341 in FIG. 3A) or using an ultrasonic cleaner (such as ultrasonic cleaner 342 in FIG. 3A).

After cleaning, the article is passed to preparation and packaging area 350 via washers 345 or pass-through window 346. After the article is packaged and otherwise prepared for sterilization, the article is then passed to sterilization area 360. Also as noted, the article is sterilized in sterilization area 360 using a sterilization device, such as high-temperature steam sterilizer 365. However, the article may be sterilized using other methods such as using a low-temperature sterilizer (such as low-temperature sterilizer 362 in FIG. 3A) which uses processes such as Ethylene Oxide (EtO), Vaporized Hydrogen Peroxide (VHP) or other low temperature sterilization methods. Upon completion of the sterilization cycle and after cooling, sterilized articles are transported for use or storage through a segregated vestibule such as sterilization area vestibule 368.

FIG. 4 shows a high-level view of sterilization processing facility 300 including the internal walls of sterilization processing facility 300. As shown in FIG. 4, decontamination area 340 is physically segregated from sterilization area 360 and from preparation and packaging area 350. Segregation between decontamination area 340 and the rest of facility 300 is due to walls, such as walls 380, 381 and 382, and screen 485. However, the combination of walls and screen are just exemplary, and any other combination of walls and screens may be used to segregate decontamination area 340 from clean areas of facility 300. However, to prevent clean areas like preparation and packaging area 340 and sterilization area 360 from being contaminated by, for example, air from decontamination area 340, decontamination area 340 must also be isolated from the clean areas. In other words, dirty air from decontamination area 340 should be prevented from flowing into preparation and packaging area 340 or sterilization area 360 in accordance with guidelines such as the Facilities Guideline Institute (FGI) guidelines for Design and Construction of Health Care Facilities and the ANSI/AAMI ST79 Guide for Steam Sterilization.

Walls 380, 381 and 382 are contiguous from wall to wall and from each wall to the floor and ceiling of shelter 102. In other words, the walls are sealed and air tight such that no air from decontamination area 340 may pass through or around the walls to sterilization area 360 or preparation and packaging area 350. However, screen 485 interrupts the contiguous line of walls. While screen 485 may comprise an air tight fabric such as plastic, it must allow a person to walk between decontamination area 340 and preparation and packaging area 350, for example, and therefore must have an opening.

As noted, walls within the interior of facility 300, such as walls 380, 381 and 382, are contiguous and sealed from wall to wall and between each wall and the ceiling and floor of the facility. However, shelter 102 includes three different heights. As shown in FIGS. 2B and 2C, main shelter body 210, expandable shelter body 220 and expandable shelter body 230 each have a different height because, as noted, expandable shelters 220 and 230 collapse into main shelter body 210. As shown by arrows in FIG. 2C, the ceilings of main shelter body 210, expandable shelter body 220 and expandable shelter body 230 sit adjacent to each other when shelter 102 is in a collapsed state. Therefore, extensions, or air lock panels, attached to the top of external or internal walls or integrated into the ceiling of shelter 102 may be used to make up for the difference in heights between the three portions of shelter 102 and to keep the walls from leaking air between the facility's different internal areas and between the internal areas and the outside.

To prevent dirty, contaminated air from passing from decontamination area 340 to preparation and packaging area 350, which would contaminate the clean air in preparation and packaging area 350 and sterilization area 360, the air pressure in each area can be monitored and controlled. As noted, decontamination HVAC 349 provides climate control to decontamination area 340 and HVAC 349 provides climate control to sterilization area 360 and/or other clean areas of sterilization processing facility 300. Furthermore, HVAC 349 and HVAC 371, which service dirty and clean portions of facility 300, respectively, are separated to prevent contamination of sterilization area 360 with contents or air from decontamination area 340. To control air flow between decontamination area 340 and preparation and packaging area 350, the air pressure must be sufficiently higher in the preparation and packaging area 350 as compared to the air pressure in the decontamination area 340 such that if air flows between the two areas, the air will flow from preparation and packaging area 350 to decontamination area 340. In other words, the decontamination area must have a negative air pressure with respect to the clean preparation and packaging area to prevent dirty air from flowing from a decontamination area to a preparation and packaging area. Furthermore, as noted, the walls within facility 300 are sealed such that air may not escape each area and HVAC 349 and 371 may control the air pressure in their respective areas. Therefore, the combination of sealed walls and control of the difference in air pressure between decontamination (“dirty”) areas and preparation and packaging or sterilization (“clean”) areas allows sterilization processing facility 300 to isolate the dirty areas from the clean areas.

Referring back to FIG. 3A, as noted, an article, such as a medical device, is sterilized in sterilization area 360 using a sterilization device, such as high-temperature steam sterilizer 365. As shown in FIG. 3A, high-temperature steam sterilizer 365 is contained in housing 366. Housing 366 may be insulated and air tight such that heat (which may be extreme), debris or other climate conditions created and/or released by sterilizer 365 is contained and not released into the rest of sterilization area 360. In other words, housing 366 thermally contains sterilizer(s) 365. If such heat, for example, were released into sterilization area 360, sterilization HVAC 371 may struggle to contain and control the air pressure and other climate conditions (e.g. temperature, humidity, filtration, etc.) within sterilization area 360. If HVAC 371 is unable to control the climate within sterilization area 360, then the air pressure in the clean areas of sterilization processing facility 300 may drop below the air pressure of decontamination area 340, causing the sterilization area to become contaminated.

HVAC area 375 which, as noted, contains steam generators 370 to provide steam to steam sterilizer 365, may also contain a device, such as a ventilation system (not shown), to remove the heat building up within area 375. HVAC ducting 372 provides conditioned air to the preparation 350 and sterilization 360 clean areas. Housing 366 may also contain a device, such as a ventilation system (not shown), to remove the heat building up within housing 366 to provide steam to steam sterilizer 365 and/or remove heat from housing 366. However, the configuration of HVAC area 375, HVAC ducting 372, sterilizer 365 and housing 366 are exemplary only and may be organized in a variety of different ways depending on the application of sterilization processing facility 300.

Vestibules 338 and 368, as noted, are used for ingress and egress from sterilization processing facility 300. Similar to other portions of facility 300, vestibules 338 and 368 must be tailored such that facility 300 meets ANSI/AAMI ST79 Guide for Steam Sterilization guidelines. Therefore, vestibules 338 and 368 include walls that are sealed/air-locked to prevent movement of air through or around the walls and that are moveable to convert shelter 102 into its collapsed state and facility 300 into its mobile layout.

FIG. 5 shows, similar to FIG. 3B, sterilization processing facility 300 in its mobile, or transportable (“packed-up”), layout. Sterilization processing facility 300 is in its mobile layout when shelter 102, in which sterilization processing facility 300 is housed, is in its collapsed state. Tractor 101 (not shown in FIG. 5) includes sets of wheels 106 and 107 and axles 586 and 587. Wheels 106 and 107 and axles are located towards the rear of tractor 101 and just below the front end of shelter 102. Shelter 102 includes sets of wheels 108 and 109 and axles 588 and 589, which are located towards the rear of shelter 102.

The Federal truck size and weight requirements are such that a semi-trailer truck, such as truck 100 shown in FIG. 1, has a specified maximum weight (e.g., 80,000 lbs.), of the truck and the cargo it carries, unless a state permit is obtained to allow for a higher weight. As noted, in its mobile layout, mobile sterilization processing facility 300 includes certain devices used in exemplary embodiments for sterilization processing, such as steam sterilizer(s) 365 and washer(s) 345. However, as shown in FIG. 3B and as noted, some devices are removed from facility 300 when the facility 300 is converted from its operational layout to its mobile layout. Removal of such devices may be necessary because, in some embodiments, the maximum weight of the non-divisible load and truck must be removed to be permitted US state department of transportations (DOTs).

The Federal truck size and weight requirements allow permitting based on retaining the commercial use/value or physical integrity of the facility. This portion of the cargo is referred to as the non-divisible load and therefore cannot be removed from the cargo without compromising the intended use of the facility. Therefore, for example, to transport the mobile sterilization processing facility 300, Federal truck size and weight regulations require that the weight of the devices of the facility that are required to retain the commercial use/value of the facility be included in the weight calculation which must be within the permittable weight limit as deemed by the US State DOTs. Therefore, certain elements of sterilization processing facility 300 that are not required to retain the commercial use/value of the facility, referred to as divisible loads, may be removed from the mobile layout of the facility (when the shelter is in its collapsed state). Divisible loads include, for example, loading carts 343, transfer carts 351, loading carts 361, transfer carts 367, sinks 341, and workstations 352. However, even if a component of mobile sterilization processing facility 300 is not required under the Federal truck size and weight requirements, it may still be practically or desired required for use of the facility in certain ways or by certain users. As noted, such removed items may therefore travel in a separate vehicle (e.g. chase vehicle) as the non-divisible load portions of the facility.

The inventor(s) have developed a unique apparatus that includes combinations of components of the sterilization processing facility 300 that are included in the mobile layout of the facility, have a combined weight that is deemed by the Federal truck size and weight requirements as an appropriate set of non-divisible components (and thus can be permitted to be transported) and does not exclude any components that Federal truck size and weight requirements deems required to be included in the non-divisible load. For example, that combination of components may include, for example, as shown in FIG. 5, steam sterilizer 365, washer/disinfector 345, water treatment system 344, among other devices. Furthermore, the internal walls within facility 300, such as walls 380, 381 and 382 as shown in FIG. 4, are removable and/or may be made of a lighter material such as, for example, aluminum instead of a heavier metal to reduce the weight of the cargo and allow for more sterilization device weight to be carried. However, various combinations of devices from sterilization processing facility 300 exist that fulfill these requirements (are included in the mobile layout of the facility, have a combined weight deemed by the Federal truck size and weight requirements as an appropriate set of non-divisible components and can be permitted to be transported) and have been contemplated by the inventors.

Furthermore, the Federal standards also require that the weight carried in shelter 102, including facility 300 in its mobile layout, must be distributed across the truck's axles, such as for example axles 586, 587, 588 and 589, evenly. For example, the regulations require that each axle only be required hold a certain amount of weight. In other words, the construction of facility 300 within shelter 102 allows for the truck's axles to properly support the load it carries. The inventors have developed a unique combination and placement of equipment within the mobile layout of facility 300, in both the longitudinal and lateral directions as shown in FIG. 5, such that the truck and its axles will support the weight of the facility while traveling. For example, an exemplary combination and placement of equipment is illustrated by the layout of equipment within the shelter 102 shown in FIG. 3.

The technology described and claimed herein is not to be limited in scope by the specific preferred embodiments herein disclosed, since these embodiments are intended as illustrations, and not limitations, of several aspects of the technology. Any equivalent embodiments are intended to be within the scope of this technology. Indeed, various modifications of the technology in addition to those shown and described herein will become apparent to those skilled in the art from the foregoing description. Such modifications are also intended to fall within the scope of the appended claims.

What is claimed is:

1. An expandable mobile sterilization shelter, comprising:
a main, non-expandable shelter body and one or more expandable shelter bodies movable relative to the main shelter body between collapsed and expanded states in which the shelter has relatively less and more available enclosed space, respectively; and
a sterilization facility including a decontamination area, processing and packaging area and sterilization area, and sterilization devices for use in each of the sterilization facility areas, wherein the facility and a first set of sterilization devices form a nondivisible load,
wherein the mobility and position of the first set of sterilization devices in the facility are such that the weight of the facility is distributed across the main and one or more expandable shelter bodies when the facility is in a mobile layout when the shelter is in its collapsed state, and when the facility is in its operational layout when the shelter is in its expanded state.
